(19) United States
(12) Patent Application Publication
(10) Pub. No.: US 2009/0305288 A1
(43) Pub. Date: Dec. 10, 2009

(54) METHODS FOR AMPLIFYING NUCLEIC ACIDS AND FOR ANALYZING NUCLEIC ACIDS THEREWITH

(75) Inventors: Mari NAKAMOTO, Tokyo (JP); Tomonori NAGAOKA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/487,201

(22) Filed: Jun. 18, 2009

Related U.S. Application Data
(63) Continuation of application No. PCT/JP2007/072292, filed on Nov. 16, 2007.

(30) Foreign Application Priority Data
Dec. 21, 2006 (JP) P2006-344006

(51) Int. Cl. C12P 19/34; C12Q 1/68

(52) U.S. Cl. 435/6; 435/91.2

(57) ABSTRACT

An object of the present invention is to control the increase in amplification errors generated during the nucleic acid amplification and thus to obtain an amplification product having a good reproducibility. The present invention is characterized in that a target nucleic acid to be amplified is amplified through a two-stage amplification process in which the amplification only of a single strand is first performed and a strand which is complementary to its amplified product is then amplified. The amplification uses a first primer which is employed for the first-stage amplification and a second primer which is employed for the second-stage amplification. These primers are each used separately, or alternatively designed to have a different stringency and used at the same time.

### TECHNICAL FIELD

The present invention is directed to methods for amplifying nucleic acids and for analyzing nucleic acids therewith. More specifically, the present invention is directed to methods for amplifying trace amounts of template nucleic acids and for analyzing nucleic acids therewith, employing a two-stage amplification process.

### BACKGROUND ART

Amplification of target nucleic acids in advance is required in order to make researches, such as gene analysis, with only small amounts of nucleic acid samples as a target. PCR processes are a technique which is capable of making effective use as an approach for such a purpose. In PCR processes, however, there is an intrinsic problem of the occurrence of amplification errors. When the occurrence of an amplification error takes place at early stages of the amplification, the error is also amplified in geometric progression and will be present in a substantial portion of the amplified products.

As a type of errors, there may be generated mismatches in base pairing. Also included are errors in cases of imbalanced amplifications taking place, for example, in which when two regions are amplified at the same time to compare the amount of their amplification products, only one of the two regions is amplified in excess (the other is less amplified), thereby resulting in lost ratios of amounts for the two regions. In addition, in cases where a template is composed of repeats of a unit sequence, as in a microsatellite region within a genome, stutter bands having shorter lengths than its native length may also appear.

In general, the amount of template required in PCR processes is in the range of several nanograms to twenty nanograms or so, and when only those amounts or less are available, it is necessary to carry out preliminary amplification in order to increase the amount of template. Processes for this purpose include, for example, a PEP (Primer Extension Pre-Amplification) process (Non-Patent Document 1), a DOP-PCR (Degenerate Oligonucleotide-Primed PCR) process (Non-Patent Document 2), and a GenomiPhi process.

In the PEP process disclosed in the Non-Patent Document 1, the amplification is performed employing a 15-mer amplification primer which is completely randomized. This process involves 50 successive thermal cycles consisting of: (1) a step of denaturing at 92° C.; (2) a step of hybridization at 37° C.; (3) a step of increasing the temperature gradually at a rate of about 0.1° C./sec from the hybridization temperature up to 55° C.; and (4) a step of performing a polymerase extension reaction at 55° C. for 4 minutes. The PEP process, which use a randomized primer, can be also applied to cases of targets having unknown sequences, but results in amplification of internal regions of the products amplified in the previous cycle or cycles. Thus, the PEP process is characterized by providing the result that there are accumulated products which become shorter in length as the thermal cycle progresses.

DOP-PCR processes enable one to amplify the sequence of a portion represented statically in an unknown template DNA. These processes employ partially-degenerative primers which bind to various sites throughout a genome. That is, these processes use amplification primers which have particular sequences at the 5′ and 3′ ends (with a statically representing 6-base degenerative segment located on the 3′ side) and a random hexamer region in the central part. In the DOP-PCR process described in the Non-Patent Document 2, the amplification is performed under a slightly stringent condition for the first five thermal cycles, and under a more stringent condition for the next thirty-five thermal cycles and at a higher annealing temperature, and is set such that during these cycles, only a primer which is completely complementary can bind to the target DNA to be amplified. However, this technology also causes deviated amplifications to take place, and results in events in which some of the genome segments are not contained in the final products.

In the PEP and DOP-PCR processes described above, since the total number of PCR cycles is increased in both cases, the degree of amplification of errors will be increased in geometric progression with the number of cycles of PCR, as in the usual PCR process. Processes which are resistant to such influences include an MDA (Multiple Displacement Amplification) process. In a version of this process, a GenomiPhi process, Phi 29 DNA polymerase is employed to perform a strand displacement amplification, thereby making it possible to carry out a non-specific and random amplification of a single-/double-stranded DNA template. However, there is a problem of the reaction time being long, and the problem of non-specific amplification is not eliminated completely.

Thus, there are available several methods in which preliminary amplification of a template nucleic acid is performed, while there is an intrinsic problem of the fact that they often cannot ensure the uniformity and quantitativeness of amplification in association with an increased number of PCR cycles, etc. and it is difficult to obtain analytical results reflecting the original condition of a genome.

[Patent Document 1] U.S. Pat. No. 6,124,120 specification
[Patent Document 2] U.S. Pat. No. 6,365,375 specification
[Patent Document 3] U.S. Patent Publication No. 2002/0160404 specification [Non-Patent Document 1] H. Telenius et al., Genomics, 1992, Vol. 13, p. 718-725

### [Non-Patent Document 2] L. Zhang et al., Proceeding of National Academy of Science, USA, 1992, Vol. 89, p. 5847-5851

### DISCLOSURE OF THE INVENTION

### Problems to be Solved by the Invention

In the present invention, it is an object to solve such problems and to reduce errors and deviations in the amplification as much as possible, thereby providing a method for performing a more accurate amplification.

### Means for Solving the Problems

Taking the above-described object into consideration, the present invention is characterized in that the present invention is configured as follows.

(1) a method for amplifying a nucleic acid, comprising:

a complementary strand amplifying step, which is carried out using a target double-stranded nucleic acid to be amplified and a first primer, wherein the first primer is complementary to a region in one strand of said nucleic acid;

a second-primer adding step, wherein a second primer is added which is complementary to a region on the 3′ side of the amplified product from said complementary strand amplifying step; and

a double strand amplifying step, wherein said target double-stranded nucleic acid to be amplified is amplified in the presence of the first primer and the second primer.

(2) a method for amplifying a nucleic acid, comprising:

a complementary strand amplifying step, which is carried out using a target single-stranded nucleic acid to be amplified and a first primer, wherein the first primer is complementary to a region in said nucleic acid;

a second-primer adding step, wherein a second primer is added which is complementary to a region on the 3′ side of the amplified product from said complementary strand amplifying step; and

a double strand amplifying step, wherein said target nucleic acid to be amplified is amplified in the presence of the first primer and the second primer.

(3) a method for amplifying a nucleic acid, comprising:

an amplification preparing step, comprising mixing a target double-stranded nucleic acid to be amplified, a first primer, and a second primer, wherein the first primer is complementary to a region in one strand of said nucleic acid, and wherein the second primer is complementary to a region in the other strand of said nucleic acid and its optimal stringency is significantly milder than that of the first primer;

a first amplification step, which is carried out under conditions having an optimal stringency for the combination of the first primer and the target nucleic acid to be amplified; and

a second amplification step, which is carried out under conditions having an optimal stringency for the combination of the second primer and the target nucleic acid to be amplified.

(4) a method for amplifying a nucleic acid, comprising:

an amplification preparing step, comprising mixing a target single-stranded nucleic acid to be amplified, a first primer, and a second primer, wherein the first primer is complementary to a region in said nucleic acid, and wherein the second primer is complementary to a region on the 3′ side of the extension product extended with said first primer and its optimal stringency is significantly milder than that of said first primer;

a first amplification step, which is carried out under conditions having an optimal stringency for the combination of the first primer and the target nucleic acid to be amplified; and

a second amplification step, which is carried out under conditions having an optimal stringency for the combination of the second primer and the amplification product of the first amplification step.

(5) the method for amplifying a nucleic acid according to (3) or (4) described above, wherein the stringency relates to the annealing temperature of the primers.
(6) the method for amplifying a nucleic acid according to (5) described above, wherein the temperature difference between the optimal annealing temperature of the first primer (T1) and the second primer (T2) is 5 to 30° C.
(7) the method for amplifying a nucleic acid according to any one of (1) to (4) described above, further comprising:

a step of quantifying an amplified product of said double-strand amplifying step or said second amplification step.

(8) the method for amplifying a nucleic acid according to (7) described above, wherein the quantifying is carried out based on a detectable label affixed in advance to at least one of said first primer and said second primer.
(9) the method for amplifying a nucleic acid according to (7) described above, wherein the quantifying is carried out by:

labeling in advance one member of a binding pair to at least one of said first primer and said second primer and adding an enzyme, the enzyme being coupled to the other member of the binding pair, to the amplified product of said double-strand amplifying step or said second amplification step, thereby forming a conjugate of the binding pair and the amplified product;

performing a reaction with said enzyme by contacting with said conjugate a substrate for said enzyme to which the detectable label is coupled; and

additionally detecting said label in the reaction product by means of said enzyme.

(10) the method for amplifying a nucleic acid according to any one of (1) to (4) described above, wherein the target nucleic acid to be amplified is selected from the group consisting of sequences having higher-order structures, sequences having GC contents equal to or higher than 50 v %, STR sequences, and microsatellite sequences.
(11) the method for amplifying a nucleic acid according to any one of (1) to (4) described above, wherein the first primer is of plural types.
(12) the method for amplifying a nucleic acid according to any one of (1) to (4) described above, wherein the amount of the target nucleic acid to be amplified is in the range of 0.1 to 5 ng prior to the amplification.
(13) a method for analyzing a nucleic acid, characterized in that the detection of the nucleic acid is carried out after amplifying the nucleic acid employing the method for amplifying the nucleic acid according to any one of (1) to (12) described above.
(14) the method for analyzing a nucleic acid according to (13) described above, characterized in that the target nucleic acid to be amplified is for LOH analysis, detection of methylation, or detection of heteroplasmy.

### EFFECTS OF THE INVENTION

The methods for amplifying a nucleic acid according to the present invention make it possible to prevent effectively amplification errors which may be generated during the amplification from being amplified in geometric progression, and thus to amplify nucleic acids which can be subjected to analyses requiring quantitativeness.

### BEST MODE FOR CARRYING OUT THE INVENTION

A method for amplifying a nucleic acid of the present invention comprises:

a complementary strand amplifying step, in which a target double-stranded nucleic acid to be amplified, and a first primer complementary to a region in one strand of said nucleic acid are used;

a second-primer adding step, in which a second primer complementary to a region on the 3′ side of the amplified product from said complementary strand amplifying step is added; and

a double strand amplifying step, in which said target double-stranded nucleic acid to be amplified is amplified in the presence of the first primer and the second primer.

Although the target nucleic acid to be amplified is not limited specifically in the practice of the present invention, it is desirable that in order for the amplification to progress with effect, the target nucleic acid to be amplified is a nucleic acid which is purified as much as possible and which does not contain any contaminants that have adverse effects on the amplification reaction. The amount of a target nucleic acid to be amplified is in the range of 0.1 to 5 ng, and more preferably 1 to 3 ng. The length of a target nucleic acid to be amplified also is not limited specifically. When genomic DNA is used as a target, however, it is desirable that treatments for fragmentation are performed in advance, such as sonication and DNase I digestion. It is preferable that the length of nucleic acids after the fragmentation is 500 bp or so.

In the complementary strand amplifying step, the amplification of one strand of the target double-stranded nucleic acid to be amplified is carried out. To this end, a first primer is prepared which has a sequence complementary to a region in the strand, and an extension reaction is performed using a polymerase. The complementary strand amplifying step is essentially a PCR process which is carried out using only a one-sided primer. Therefore, the first primer can be prepared using known methods, and it is desirable to employ, as a polymerase, polymerases used in the usual PCR and which can be used in thermal cycles. The complementary strand amplifying step uses buffers, other necessary substrates (dNTPs), and the like, which are suitable for reactions for the amplification.

The complementary strand amplifying step consists of the following three sub-steps:

(1) a denaturing step, in which the target nucleic acid to be amplified is degenerated;
(2) an annealing step, in which the first primer and the target nucleic acid to be amplified are annealed; and
(3) an extension step, in which the extension reaction of the first primer annealed to the target nucleic acid to be amplified is carried out.

It is preferable that the complementary strand amplifying step consisting of these three sub-steps is carried out at a number of cycles in the range of 20 to 40 rounds, because less than 20 rounds will result in a reduced degree of amplification of the complementary strand and more than 40 rounds will get rise to the tendency to inhibit the reaction in the double-strand amplifying step described below.

The denaturing step is not limited in particular, if the denaturing is at temperatures that ensure that the denaturing of the target nucleic acid to be amplified is achieved. However, it is desirable that this step is carried out at temperatures around 95° C. for 10 minutes or so, in order to ensure that the denaturing of a double-stranded nucleic acid is achieved. The annealing step is carried out under optimal conditions (temperature, salt concentration, etc.), which are determined as appropriate by those skilled in the art, depending upon the length of base pairing between the first primer and the target nucleic acid to be amplified, the GC content of the base pairs, and the like. When the length of the first primer is in the range of 15 to 25 bases, it is possible, in ordinary cases, that annealing is performed in the range of 50 to 65° C. for a period of 30 seconds to 1 minute, because such annealing could form a hybrid consisting only of specific base pairs between the primer and the target nucleic acid to be amplified, without non-specific bonding between them. The final extension step is carried out by changing the temperature of the reaction system from the annealing temperature up to a temperature suitable for the polymerase used and keeping the reaction system at that temperature. The period for which the reaction system is kept is a period sufficient for the primer to be extended, by the extension reaction, to a necessary and sufficient length, that is, a period for which the first primer is extended including a region which is recognized and bound by the second primer in the double strand amplifying step after adding the second primer. This period can be determined as appropriate by those skilled in the art, based upon information about the distance between the respective regions in a nucleic acid recognized by the first primer and the second primer, a typical reaction rate of a polymerase used, and others. In most instances, the reaction rate of polymerases is on the order of 1 kb/min, and thus as the extension time (in min) could be set a value of the length required by the extension (in kb) divided by the reaction rate.

After the complementary strand amplification step has been completed, the second-primer adding step is carried out, wherein a second primer is added which is complementary to a region on the 3′ side of the amplified product from said complementary strand amplifying step. The second primer is also prepared by known methods, but is produced so as to have a complementary region on the 3′ side of the amplified product from said complementary strand amplifying step, as mentioned above. When the addition of the second primer requires that the buffer be adjusted, the buffer is adjusted as appropriate, so that the amplification step described below is not inhibited.

Subsequently, the double strand amplifying step is carried out, wherein said target double-stranded nucleic acid to be amplified is amplified in the presence of the first primer and the second primer. In this step, the second primer which recognizes the complementary strand amplified by the first primer first results in the amplification of a strand which is complementary to that complementary strand, and the usual PCR amplification is caused with the first and second primers, if an excess of the first primer is present, which has not been used in the complementary strand amplifying step described above. As in the usual PCR, the two strands are amplified in geometric progression. Also in this step of amplifying the double strand, thermal cycles of denaturing, annealing, and extending steps are performed as in the complementary strand amplifying step described above. The number of cycles can be determined as appropriate by those skilled in the art, taking into consideration the amount of an extension product extended in the complementary strand extending step, the amount of a double-stranded nucleic acid required in the end, the amplification efficiency in each of the steps, and the like. Small numbers of amplification cycles will result in insufficient amounts of amplification and thus do not allow one to make a high-reliability analysis, and on the other hand, excessive numbers of amplification cycles will lead to increased errors of amplification and thus do not allow one to make a quantitative analysis. For these reason, it is more preferable that when the amount prior to the amplification of a target nucleic acid to be amplified is 0.1 to 5 ng, the number of cycles of amplification is set, more specifically, to be in the range of 20 to 35 cycles.

As mentioned above, the present invention results in the target nucleic acid to be amplified being amplified (in the complementary strand amplifying step) in arithmetic progression, not in geometric progression, by carrying out in advance the complementary strand amplifying step. Errors contained in the amplified products depend on reaction conditions and polymerases used, but in the complementary strand amplifying step, arithmetical or linear amplification is achieved in stead of geometrical amplification, and thus the degree to which inevitable errors are amplified also remains under arithmetical or linear amplification. More specifically, in the case where a double-stranded template is amplified at the same time by conventional PCR processes, if the number of cycles of amplification is 40 cycles, then an error generated in the first amplification stage will become approximately 2⁴⁰ times after the 40 cycles (with the assumption that the amplification efficiency of each cycle is 100%). When the complementary strand amplifying step in the method of the present invention is carried out, on the other hand, a one-sided strand which has been amplified does not serve as a temperate for the next amplification, and thus is not carried over into the subsequent amplification. (However, an error generated in a one-sided strand which has been amplified is amplified in a geometric progression with the number of PCR cycles in the PCR reaction after the complementary strand amplifying step.) In conventional PCR, errors are contained in about one fourth of the whole amplified product. In the complementary strand amplifying step of the present invention, on the other hand, errors generated in the amplified products are at a certain percentage specific to the amplification system. These two approaches are different in the amount of amplifications, and therefore, upon consideration of this difference, the number of strands containing errors is much larger in conventional PCR than in the complementary strand amplifying step of the present invention. As a result, there will be high probabilities of making an analysis based on errors when sequences are analyzed based on conventional PCR amplification products. In the present invention, on the other hand, since the number of strands containing errors is at a certain percentage which is very small, it would be possible, by performing, as appropriate, an additional amplification of a nucleic acid with the usual amplification process, not only to provide sufficient amounts of nucleic acid necessary for analysis, but also to reduce the percentage and probability of errors contained in the resultant nucleic acid to a sufficiently low degree.

Although the preceding paragraphs has described the present invention in cases where a target nucleic acid to be amplified is a double strand, it is also possible, when a target nucleic acid to be amplified is a single strand, to amplify small amounts of nucleic acid by essentially similar processes. That is, regarding to a target single-stranded nucleic acid to be amplified, it is possible to amplify the nucleic acid in arithmetic progression, without geometrical amplification of errors, by carrying out a method comprising:

a complementary strand amplifying step, which is carried out using a target single-stranded nucleic acid to be amplified and a first primer, wherein the first primer is complementary to a region in said nucleic acid;

a second-primer adding step, wherein a second primer is added which is complementary to a region on the 3′ side of the amplified product from said complementary strand amplifying step; and

a double strand amplifying step, wherein said target nucleic acid to be amplified is amplified in the presence of the first primer and the second primer.

The present method of amplifying a nucleic acid can be a method which comprises:

an amplification preparing step, comprising mixing a target double-stranded nucleic acid to be amplified, a first primer, and a second primer, wherein the first primer is complementary to a region in one strand of said nucleic acid, and wherein the second primer is complementary to a region in the other strand of said nucleic acid and its optimal stringency is significantly milder than that of the first primer;

a first amplification step, which is carried out under conditions having an optimal stringency for the combination of the first primer and the target nucleic acid to be amplified; and

a second amplification step, which is carried out under conditions having an optimal stringency for the combination of the second primer and the target nucleic acid to be amplified.

In the amplification preparing step described above, two types of primers whose stringency is significantly different in relation to a target nucleic acid to be amplified are mixed with a doubled-stranded nucleic acid which is a target to be amplified. Subsequently, the first amplification step is carried out under conditions having an optimal stringency for the first primer and the target nucleic acid to be amplified, and the second amplification step is then carried out under conditions having an optimal stringency for the second primer and the target nucleic acid to be amplified.

Stringency conditions in the first amplification step are of stringency which is optimal to the first primer and the target nucleic acid to be amplified and which is significantly severer than that optimal to the second primer and the target nucleic acid to be amplified, thereby resulting in amplification only between the first primer and the target nucleic acid to be amplified. Here, the stringency can be related, for example, to the annealing temperature of the primers. In this case, it is preferable that the difference between the optimal annealing temperature of the first primer (T1) and the second primer (T2) is 5 to 30° C. More preferably, the difference between T1 and T2 is 10 to 15° C.

Next is the practice of the second amplification step. The second amplification step is carried out under conditions having an optimal stringency for the combination of the second primer and the target nucleic acid to be amplified, which stringency is significantly milder than that of the first amplification step. The first amplification step results in the occurrence of a reaction using the first primer, and the second amplification step results in the occurrence of the usual PCR with the first primer and the second primer. Therefore, it is desirable that the complete consumption of the first primer is not reached in the first amplification step.

Although the preceding paragraphs has described the present invention in cases where a target nucleic acid to be amplified is a double strand, it is also possible, when a target nucleic acid to be amplified is a single strand, to amplify a nucleic acid by essentially similar processes. That is, in this case, there can be provided a method for amplifying a nucleic acid, the method comprising an amplification preparing step, comprising mixing a target single-stranded nucleic acid to be amplified, a first primer, and a second primer, wherein the first primer is complementary to a region in said nucleic acid, and wherein the second primer is complementary to a region on the 3′ side of an extension product extended with said first primer and its optimal stringency is significantly milder than that of the first primer; a first amplification step, which is carried out under conditions having an optimal stringency for the combination of the first primer and the target nucleic acid to be amplified; and a second amplification step, which is carried out under conditions having an optimal stringency for the combination of the second primer and the amplification product of the first amplification step.

In the present invention, the practice of the above-described amplification method can be followed by quantification of its amplified product. Specifically, the amount of an amplified product in the above-described double strand amplifying step or the above-described second amplification step is quantified. Methods for quantification can include those which directly quantify an amplification product itself, and those which indirectly quantify a physical property's value which is proportional to the amount of an amplification product. Direct quantification methods can include those methods which quantify a detectable label, such as a fluorescent label introduced into the primer in advance. Indirect quantification methods include detection with an intercalator, such as SYBR Green.

Another type of indirect quantification methods can include a method in which quantifying is carried out by: labeling in advance one member of a binding pair to at least one of said first primer and said second primer and adding an enzyme, the enzyme being coupled to the other member of the binding pair, to the amplified product of said double-strand amplifying step or said second amplification step, thereby forming a conjugate of the binding pair and the amplification product; performing a reaction with said enzyme by contacting with said conjugate a substrate for said enzyme to which the detectable label is coupled; and additionally detecting said label in the reaction product by means of said enzyme.

The methods for amplifying a nucleic acid according to the present invention can be suitably employed in cases where a target nucleic acid to be amplified represents a sequence having a higher-order structure, a sequence having a GC content equal to or higher than 50%, more preferably 60%, an STR sequence, or a microsatellite sequence, because these sequences generally tend to generate errors during the amplification, and in consequence, are likelier to cause the occurrence of errors at early stages when these sequences are amplified using the usual PCR. The present invention has a remarkably low percentage, in the whole amplification product, of products containing such errors, compared with amplification products obtained when amplification methods with the usual PCR are applied. Therefore, the advantage of carrying out the present method is brought about when a target nucleic acid to be amplified contains any of these sequences.

In methods for amplifying a nucleic acid according to the present invention, it is possible to use plural types of first primer. In other words, it is possible to prepare primers, each of the primers recognizing one of plural regions in one strand of a target nucleic acid to be amplified, so that the amplified products having eventually different lengths are obtained. Since all the regions in the sequence of a target nucleic acid to be amplified are not always amplifiable in an equal manner, it is possible that when the initial amount of a target nucleic acid to be amplified is extremely small, the probability of amplifying a target to be amplified is increased, according to the present amplification method, by amplifying plural regions at the same time in this way.

Sequences prone to cause amplification errors, such as sequences having higher-order structures, sequences having high GC contents, STR sequences, and microsatellite sequences, are difficult to amplify with the usual PCR and have the tendency to lose the quantitativeness upon increasing the number of cycles of PCR. Even in these cases, it is possible that these sequences are detected with a high degree of quantitativeness, by employing the methods for amplifying a nucleic acid according to the present invention. In addition to this, the present methods could be also employed suitably for LOH analysis, detection of methylation, detection of heteroplasmy, and others.

An epigenetic analysis in canceration is, in some cases, to compare the degree of methylation in respective tissues. In doing this, quantitative analysis is required because it is necessary to make an accurate comparison of the degree of methylation. When the amount of genome is small in making a methylation analysis employing PCR, the number of cycles of PCR must be usually increased. Simply increasing the number of cycles will lead to amplification of errors and thus does not allow one to make a quantitative analysis. By applying the present invention and performing pre-amplification once, however, a quantitative analysis will be permitted.

Mutations in mitochondrial DNA may cause diseases. The severity of these diseases relies on how mutations take place, the ratio of mutant mitochondrial DNA to wild-type DNA in cells, and the like. In order to understand diseases caused by mutations in mitochondrial DNA, therefore, it is important to determine the percentage of heteroplasmy. Also in this case, the number of cycles of PCR must be usually increased when the amount of genome is small. Simply increasing the number of cycles will lead to amplification of errors and thus does not allow one to make a quantitative analysis. By applying the present invention and performing pre-amplification once, however, a quantitative analysis will be permitted.

### Example 1

The following sample DNA and oligonucleotides were employed.

DNA: human genomic DNA (2 ng) (Promega, Human Genomic DNA: Male)

Oligonucleotides:

A second primer having a base sequence set forth in SEQ ID No. 1, D3S1293for (HEX-labeled),
A first primer having a base sequence set forth in SEQ ID No. 2, D3S1293rev.

The whole amount of the above-described human genomic DNA, 12.5 p mol of the above-described first primer, and 5 μl of 10×Ex Taq Buffer were mixed and adjusted to make a total volume of 50 μl so as for the Buffer and the dNTP mix to be at 1× and 0.2 mM, respectively. To this mixture was added 1.25 units of TaKaRa Ex Taq, and subjected to repeating 30 thermal cycles of 94° C. for 30 seconds, 55° C. for 30 seconds, and 74° C. for 30 seconds for amplifying a one-sided strand. After that, to the reaction solution were added 12.5 pmol of the above-described second primer and 1.25 units of TaKaRa Ex Taq, and PCR amplification was performed by repeating 25 thermal cycles of 94° C. for 30 seconds, 55° C. for 30 seconds, and 74° C. for 30 seconds.

The detection of amplification products was performed on Genetic Analyzer 3130x1 (ABI).

### (Results)

By carrying out the present method, there were detected bands which were not detected when the usual 25-cycle PCR was merely performed without carrying out the complementary strand extending reaction employing the first primer. In addition, a quantitative analysis (comparison of the ratio of peak intensities) was allowed to be made using the amplified products.

### Example 2

The following sample DNA and oligonucleotides were employed.

DNA: human genomic DNA (2 ng) (Promega, Human Genomic DNA: Male)

Oligonucleotides:

A second primer having a base sequence set forth in SEQ ID No. 1, D3S1293for (HEX-labeled),
A first primer having a base sequence set forth in SEQ ID No. 2, D3S1293rev,
Another second primer having a base sequence set forth in SEQ ID No. 3, D3S1234for (6-FAM-labeled),
Another first primer having a base sequence set forth in SEQ ID No. 4, D3S1234rev.

The whole amount of the above-described human genomic DNA, 12.5 pmol of each of the first primers rev's, and 5 μl of 10×Ex Taq Buffer were mixed and adjusted to make a total volume of 50 μl so as for the Buffer and the dNTP mix to be at 1× and 0.2 mM, respectively. To this mixture was added 1.25 units of TaKaRa Ex Taq, and subjected to repeating 30 thermal cycles of 94° C. for 30 seconds, 55° C. for 30 seconds, and 74° C. for 30 seconds for amplifying a one-sided strand. After that, to the reaction solution were added 12.5 pmol of each of the second primers for's and 1.25 units of TaKaRa Ex Taq, and PCR amplification was performed by repeating 25 thermal cycles of 94° C. for 30 seconds, 55° C. for 30 seconds, and 74° C. for 30 seconds. The detection of amplification products was performed on Genetic Analyzer 3130x1 (ABI).

### (Results)

By carrying out the present method, there were detected bands which were not detected when the usual 25-cycle PCR was merely performed without carrying out the complementary strand extending reaction employing the first primers. In addition, a quantitative analysis was allowed to be made using the amplified products.

### Example 3

The following sample DNA and oligonucleotides were employed.

DNA: human genomic DNA (2 ng) (Promega, Human Genomic DNA: Male)

Oligonucleotides:

A second primer having a base sequence set forth in SEQ ID No. 1, D3S1293for (HEX-labeled),
A first primer having a base sequence set forth in SEQ ID No. 2, D3S1293rev,
Another second primer having a base sequence set forth in SEQ ID No. 3, D3S1234for (6-FAM-labeled),
Another first primer having a base sequence set forth in SEQ ID No. 4, D3S1234rev.

The whole amount of the above-described human genomic DNA, 12.5 pmol of each of the first primers rev's, and 5 μl of 10×Ex Taq Buffer were mixed and adjusted to make a total volume of 50 μl so as for the Buffer and the dNTP mix to be at 1× and 0.2 mM, respectively. To this mixture was added 1.25 units of TaKaRa Ex Taq, and subjected to repeating 40 thermal cycles of 94° C. for 30 seconds, 55° C. for 30 seconds, and 74° C. for 30 seconds for amplifying a one-sided strand. After that, the reaction solution was divided into two aliquots, which were transferred into tubes 1 and 2 containing 25 μl of a new PCR reaction solution (Ex Taq Buffer, 1×; dNTP mix, 0.2 mM). To the tube 1 were added 6.25 pmol of one of the first primers D3S1293rev and 12.5 pmol of one of the second primers D3S1293for. To the tube 2 were added 6.25 pmol of the other of the first primers D3S1234rev and 12.5 pmol of the other of the second primers D3S1234for. Finally, to each of the tubes 1 and 2 was added 1.25 units of TaKaRa Ex Taq, and PCR amplification was performed by repeating 25 thermal cycles of 94° C. for 30 seconds, 55° C. for 30 seconds, and 74° C. for 30 seconds. The detection of amplification products was performed on Genetic Analyzer 3130x1 (ABI).

### (Results)

Amplified products were able to be detected for the respective tubes.

### Example 4

The following sample DNA and oligonucleotides were employed.

DNA: human genomic DNA (2 ng) (Promega, Human Genomic DNA: Male)

Oligonucleotides:

A second primer having a base sequence set forth in SEQ ID No. 5, TP53for (HEX-labeled),
A first primer having a base sequence set forth in SEQ ID No. 6, TP53rev.

The whole amount of the above-described human genomic DNA, 20 pmol of each of the above-described first and second primers, and 5 μl of 10×Ex Taq Buffer were mixed and adjusted to make a total volume of 50 μl so as for the Buffer and dNTP mix to be at 1× and 0.2 mM, respectively. To this mixture was added 1.25 units of TaKaRa Ex Taq, and subjected to repeating 30 thermal cycles of 94° C. for 30 seconds, 65° C. for 30 seconds, and 74° C. for 30 seconds for amplifying a one-sided strand only from the first primer. After that, PCR amplification was performed by repeating 25 thermal cycles of 94° C. for 30 seconds, 55° C. for 30 seconds, and 74° C. for 30 seconds. The detection of amplification products was performed on Genetic Analyzer 3130x1 (ABI).

### (Results)

By carrying out the present method, there were detected bands which were not detected when the usual 25-cycle PCR was merely performed without carrying out the complementary strand extending reaction employing the first primer. In addition, a quantitative analysis was allowed to be made using the amplified products.

### Comparative Example 1

### Amplification with GenomiPhi DNA Amplification Kit (GE Healthcare Bio-sciences K.K.)

As a sample DNA was used DNA (2 ng) extracted from a cancer tissue (paraffin-embedded sections) and subjected to fragmentation.

A DNA solution suspended in 1 μl of distilled water (or TE buffer) and 9 μl of sample buffer were mixed and subjected to thermal denaturing at 95° C. for 3 minutes, followed by rapid cooling. Then, 9 μl of reaction buffer and 1 μl of enzyme mix were mixed and incubated at 30° C. for a period of 16 to 18 hours. Finally, the enzyme was deactivated at 65° C. for 10 minutes and quantification was performed using PicoGreen® ds DNA Quantification Assay (Molecular Probes).

### (Results)

The quantification of products amplified with GenomiPhi was performed on Genetic Analyzer 3130x1 (ABI). Their quantitative results were almost the same as those of control reactions having no template. In addition, the results suggested a large amount of products resulting from non-specific amplification. The usual PCR was further performed using 20 ng of the products, but there was not obtained any amplified product in the region of interest (many non-specific signals were detected).

### Comparative Example 2

### Analysis by Increasing the Number of Cycles only with PCR

The following sample DNA and oligonucleotides were employed.

DNA: human genomic DNA, cancer tissue derived DNA (20 or 2 ng) extracted from paraffin-embedded sections

Oligonucleotides:

A primer having a base sequence set forth in SEQ ID No. 5, TP53for (HEX-labeled),
A primer having a base sequence set forth in SEQ ID No. 6, TP53rev.

20 or 2 ng of the above-described human genomic DNA, 20 pmol of each of the above-described PCR primers, and 5 μl of 10×Ex Taq Buffer were mixed and adjusted to make a total volume of 50 μl so as for the Buffer and the dNTP mix to be at 1× and 0.2 mM, respectively. To this mixture was added 1.25 units of TaKaRa Ex Taq, and subjected to repeating 25 (or 35) thermal cycles of 94° C. for 30 seconds, 65° C. for 30 seconds, and 74° C. for 30 seconds for PCR amplification. The detection of amplification products was performed on Genetic Analyzer 3130x1 (ABI).

### (Results)

Amplified products were not able to be detected when carrying out 25 thermal cycles with the template DNA set to be at 2 ng, whereas results having good reproducibility were obtained when carrying out 25 thermal cycles with the template DNA set to be at 20 ng.

Amplified products were obtained when carrying out 35 thermal cycles with the template DNA set to be at 2 ng. In comparison of the respective amplification patterns between the two tubes in a pair of two replicates, however, the respective tubes had a different ratio of the amounts of two existing peaks. This means that the reproducibility of amplification seemed to be lost.

### INDUSTRIAL APPLICABILITY

The methods for amplifying a nucleic acid according to the present invention allow one to use template nucleic acids which are present in only small amounts, in amplifying the nucleic acids in early stages of nucleic acid analyses requiring quantitativeness, due to the fact that the degree of amplification of errors can be controlled remarkably.

1. A method for amplifying a nucleic acid, comprising:
a complementary strand amplifying step, in which a target double-stranded nucleic acid to be amplified, and a first primer complementary to a region in one strand of said nucleic acid are used;
a second-primer adding step, in which a second primer complementary to a region on the 3′ side of the amplified product from said complementary strand amplifying step is added;
a double strand amplifying step in which said target double-stranded nucleic acid to be amplified is amplified in the presence of the first primer and the second primer.

2. A method for amplifying a nucleic acid, comprising:
a complementary strand amplifying step, in which a target single-stranded nucleic acid to be amplified, and a first primer complementary to a region in said nucleic acid, are used;
a second-primer adding step, in which a second primer complementary to a region on the 3′ side of the amplified product from said complementary strand amplifying step is added; and
a double strand amplifying step, in which said target nucleic acid to be amplified is amplified in the presence of the first primer and the second primer.

3. A method for amplifying a nucleic acid, comprising:
an amplification preparing step, in which a target double-stranded nucleic acid to be amplified, a first primer complementary to a region in one strand of said nucleic acid, and a second primer complementary to a region in the other strand of said nucleic acid and optimal stringency of which being significantly milder than that of the first primer are mixed;
a first amplification step, being carried out under conditions having an optimal stringency for the combination of the first primer and the target nucleic acid to be amplified; and
a second amplification step, being carried out under conditions having an optimal stringency for the combination of the second primer and the target nucleic acid to be amplified.

4. A method for amplifying a nucleic acid, comprising:
an amplification preparing step, in which a target single-stranded nucleic acid to be amplified, a first primer complementary to a region in said nucleic acid, and a second primer complementary to a region on the 3′ side of the extension product extended with said first primer and optimal stringency of which being significantly milder than that of said first primer are mixed;
a first amplification step, being carried out under conditions having an optimal stringency for the combination of the first primer and the target nucleic acid to be amplified; and
a second amplification step, being carried out under conditions having an optimal stringency for the combination of the second primer and the amplification product of the first amplification step.

5. The method for amplifying a nucleic acid according to claim 3 or 4, wherein the stringency relates to the annealing temperature of the primers.

6. The method for amplifying a nucleic acid according to claim 5, wherein the temperature difference between the optimal annealing temperature of the first primer (T1) and the second primer (T2) is 5 to 30° C.

7. The method for amplifying a nucleic acid according to any one of claims 1 to 4, further comprising:
a step of quantifying an amplified product of said double-strand amplifying step or said second amplification step.

8. The method for amplifying a nucleic acid according to claim 7, wherein the quantifying is carried out based on a detectable label affixed in advance to at least one of said first primer and said second primer.

9. The method for amplifying a nucleic acid according to claim 7, wherein the quantifying is carried out by:
labeling in advance one member of a binding pair to at least one of said first primer and said second primer, and adding an enzyme, the enzyme being coupled to the other member of the binding pair, to the amplified product of either said double-strand amplifying step or said second amplification step, thereby forming a conjugate of the binding pair and the amplified product;
performing a reaction with said enzyme by contacting with said conjugate a substrate for said enzyme to which the detectable label is coupled; and
detecting said label in the reaction product by means of said enzyme.

10. The method for amplifying a nucleic acid according to any one of claims 1 to 4, wherein the target nucleic acid to be amplified is selected from the group consisting of sequences having higher-order structures, sequences having GC contents equal to or higher than 50%, STR sequences, and microsatellite sequences.

11. The method for amplifying a nucleic acid according to any one of claims 1 to 4, wherein the first primer is of plural types.

12. The method for amplifying a nucleic acid according to any one of claims 1 to 4, wherein the amount of the target nucleic acid to be amplified is in the range of 0.1 to 5 ng prior to the amplification.

13. A method for analyzing a nucleic acid, characterized in that the detection of the nucleic acid is carried out after amplifying the nucleic acid employing the method for amplifying the nucleic acid according to any one of claims 1 to 4.

14. The method for analyzing a nucleic acid according to claim 13, characterized in that the target nucleic acid to be amplified is for LOH analysis, detection of methylation, or detection of heteroplasmy.


SEQUENCE LISTING (6 sequences)

SEQ ID NO: 1 - DNA, 18 bp, Artificial Sequence
  Fw primer (D3S1293)
actcacagagccttcaca

SEQ ID NO: 2 - DNA, 19 bp, Artificial Sequence
  Rev primer (D3S1293)
catggaaatagaacagggt

SEQ ID NO: 3 - DNA, 20 bp, Artificial Sequence
  Fw primer (D3S1234)
cctgtgagacaaagcaagac

SEQ ID NO: 4 - DNA, 20 bp, Artificial Sequence
  Rev primer (D3S1234)
gacattaggcacagggctaa

SEQ ID NO: 5 - DNA, 18 bp, Artificial Sequence
  Fw primer (TP53)
agggatactattcagccc

SEQ ID NO: 6 - DNA, 22 bp, Artificial Sequence
  Rev primer (TP53)
actgccactccttgccccattc